(19) United States
(12) Patent Application Publication
(10) Pub. No.: US 2017/0348382 A1
(43) Pub. Date: Dec. 7, 2017

(54) LYOPHILIZED INJECTABLE COMPOSITIONS OF DAPTOMYCIN

(71) Applicant: DR. REDDY'S LABORATORIES LIMITED, Hyderabad (IN)

(72) Inventors: Sushil KURADE, Hyderabad (IN); Sachin Appasaheb SURWASE, Ahmednagar (IN); Somashekhar BATTINI, Hyderabad (IN); Bhushan Subhash YEOLA, Nasik (IN); Harshal BHAGWATWAR, Hyderabad (IN)

(21) Appl. No.: 15/536,982

(22) PCT Filed: Dec. 16, 2015
(86) PCT No.: PCT/IB2015/059668

§ 371 Date: Jun. 16, 2017

(30) Foreign Application Priority Data
Dec. 16, 2014 (IN) 6338/CHE/2014
Feb. 27, 2015 (IN) 969/CHE/2015

(51) Int. Cl. A61K 38/12; A61K 9/19; A61K 9/00; A61K 9/08; A61K 47/14; A61K 47/02

(52) U.S. Cl. CPC A61K 38/12; A61K 47/14; A61K 47/02; A61K 9/0019; A61K 9/08; A61K 9/19

(57) ABSTRACT

The present invention relates to lyophilized compositions comprising daptomycin or pharmaceutically acceptable salts or solvates thereof with improved reconstitution time for parenteral administration and the processes for preparation thereof.

### FIELD OF THE INVENTION

The present invention relates to lyophilized compositions comprising daptomycin or pharmaceutically acceptable salts or solvates thereof with improved reconstitution time for parenteral administration and the processes for preparation thereof.

### BACKGROUND OF THE INVENTION

Daptomycin is a cyclic lipopeptide antibacterial agent derived from the fermentation of streptomyces roseosporus and its chemical name is N-decanoyl-L-tryptophyl-Dasparaginyl-L-aspartyl-L-threonylglycyl-L-ornithyl-L-aspartyl-D-alanyl-L-aspartylglycyl-D-seryl-threo-3-methyl-L-glutamyl-3-anthraniloyl-L-alanine ε1-lactone. Daptomycin is structurally represented as

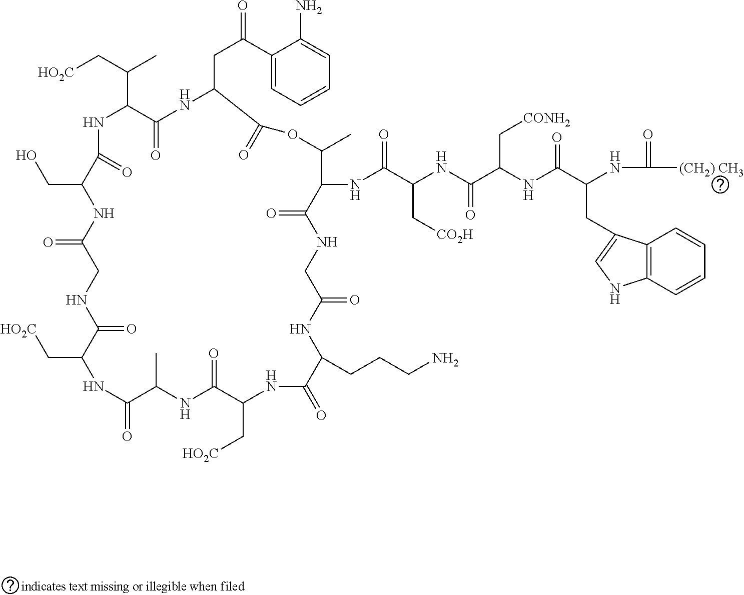

Daptomycin is used for the treatment of complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only) and Staphylococcus aureus bloodstream infections (bacteremia), including those with right-sided infective endocarditis, caused by methicillin-susceptible and methicillin-resistant isolates. U.S. Pat. No. 4,537,717 discloses the structural formula of daptomycin and the process for preparation thereof.

Daptomycin is marketed in the United States by Cubist Pharmaceuticals Inc with the trade name CUBICIN® in the form of injection containing 500 mg/vial. In Europe, CUBICIN® is marketed by Novartis Europharm as CUBICIN® powder that is made up into a solution for injection or infusion. The single vials contain 350 mg or 500 mg of daptomycin. The currently marketed products of injectable daptomycin are supplied in a single use vial as a sterile, preservative-free, pale yellow to light brown, lyophilized cake containing approximately 500 mg or 350 mg of daptomycin for intravenous (IV) use following reconstitution with 0.9% sodium chloride injection. The only inactive ingredient is sodium hydroxide, which is used in minimal quantities for pH adjustment.

For reconstitution of CUBICIN® with 0.9% sodium chloride injection, it is essential that the contents of the vial should be reconstituted using aseptic technique which is described as below.
a) To minimize foaming, AVOID vigorous agitation or shaking of the vial during or after reconstitution.
b) Remove the polypropylene flip-off cap from the CUBICIN® vial to expose the central portion of the rubber stopper.
c) Slowly transfer 10 mL of 0.9% sodium chloride injection through the center of the stopper into the CUBICIN® vial, pointing the transfer needle toward the wall of the vial.
d) Ensure that all of the CUBICIN® powder is wetted by gently rotating the vial.
e) Allow the wetted product to stand undisturbed for 10 minutes.
f) Gently rotate or swirl the vial contents for a few minutes, as needed, to obtain a completely reconstituted solution.

Thus as indicated above the major drawbacks of CUBICIN® is the long reconstitution procedure, that is about 10 minutes and is further followed by gentle rotation or swirling. Such long reconstitution time is not ideal in the therapeutic setting with respect to ease of administration.

U.S. Pat. No. 8,835,382 discloses lyophilized compositions of daptomycin with improved reconstitution time that is achieved with sugars such as sucrose and the formulation has a pH of 6.5 to about 7.5.

PCT Publication No. WO2014041425 discloses lyophilized compositions of daptomycin with improved reconstitution time that is achieved with the additives selected from the group consisting of pharmaceutically acceptable antioxidants, pharmaceutically acceptable organic acids and salts thereof, pharmaceutically acceptable glucose derivatives and salts thereof.

PCT Publication No. WO2014045296 discloses lyophilized formulation of daptomycin with improved reconstitution time that is achieved with tocopheryl phosphate hydrolysate mixture (TPM).

However, there still exists a need for alternative lyophilized daptomycin compositions that exhibit rapid reconstitution time in pharmaceutically acceptable diluents, preferably less than 10 minutes.

### SUMMARY OF THE INVENTION

The present invention relates to lyophilized injectable compositions of daptomycin or pharmaceutically acceptable salts or solvates thereof, having improved reconstitution time.

The present invention also relates to aqueous liquid composition for lyophilization comprising daptomycin or pharmaceutically acceptable salts or solvates thereof and a solvent, wherein the pH of the liquid composition is from about 4 to about 9.

The present invention further relates to aqueous liquid composition for lyophilization comprising daptomycin or pharmaceutically acceptable salts or solvates thereof, wherein the pH of the liquid composition is about 8.5.

The present invention also relates to lyophilized composition comprising daptomycin or pharmaceutically acceptable salts or solvates thereof and a preservative.

The present invention further relates to lyophilized composition comprising daptomycin or pharmaceutically acceptable salts or solvates thereof, a preservative and a pH adjusting agent.

The present invention further relates to aqueous liquid composition for lyophilization comprising daptomycin or pharmaceutically acceptable salts or solvates thereof and a preservative.

The present invention also relates to aqueous liquid composition for lyophilization comprising daptomycin or pharmaceutically acceptable salts or solvates thereof, and a preservative, wherein the pH of the liquid composition is from about 4 to about 9.

The present invention also relates to aqueous liquid composition for lyophilization comprising daptomycin or pharmaceutically acceptable salts or solvates thereof, preservative and a pH adjusting agent, wherein the pH of the liquid composition is from about 4 to about 9.

The present invention also relates to lyophilized composition comprising daptomycin or pharmaceutically acceptable salts or solvates thereof and methyl paraben.

The present invention further relates to lyophilized composition comprising daptomycin or pharmaceutically acceptable salts or solvates thereof, methyl paraben and sodium hydroxide.

The present invention further relates to aqueous liquid composition for lyophilization comprising daptomycin or pharmaceutically acceptable salts or solvates thereof and methyl paraben.

The present invention also relates to aqueous liquid composition for lyophilization comprising daptomycin or pharmaceutically acceptable salts or solvates thereof, and methyl paraben, wherein the pH of the liquid composition is from about 4 to about 9.

The present invention also relates to aqueous liquid composition for lyophilization comprising daptomycin or pharmaceutically acceptable salts or solvates thereof, methyl paraben and sodium hydroxide, wherein the pH of the liquid composition is from about 4 to about 9.

The present invention also relates to aqueous liquid composition for lyophilization comprising daptomycin or pharmaceutically acceptable salts or solvates thereof, methyl paraben and sodium hydroxide, wherein the pH of the liquid composition is about 4.7.

The present invention further relates to lyophilized composition comprising daptomycin or pharmaceutically acceptable salts or solvates thereof, preservative and a chaotropic agent.

The present invention further relates to lyophilized composition comprising daptomycin or pharmaceutically acceptable salts or solvates thereof, preservative, chaotropic agent and a pH adjusting agent.

The present invention further relates to aqueous liquid composition for lyophilization comprising daptomycin or pharmaceutically acceptable salts or solvates thereof, preservative and a chaotropic agent.

The present invention also relates to aqueous liquid composition for lyophilization comprising daptomycin or pharmaceutically acceptable salts or solvates thereof, preservative and a chaotropic agent, wherein the pH of the liquid composition is from about 4 to about 9.

The present invention also relates to aqueous liquid composition for lyophilization comprising daptomycin or pharmaceutically acceptable salts or solvates thereof, preservative, chaotropic agent and a pH adjusting agent, wherein the pH of the liquid composition is from about 4 to about 9.

The present invention also relates to lyophilized composition comprising daptomycin or pharmaceutically acceptable salts or solvates thereof, methyl paraben and sodium chloride.

The present invention further relates to lyophilized composition comprising daptomycin or pharmaceutically acceptable salts or solvates thereof, methyl paraben, sodium chloride and sodium hydroxide.

The present invention further relates to aqueous liquid composition for lyophilization comprising daptomycin or pharmaceutically acceptable salts or solvates thereof, methyl paraben and sodium chloride.

The present invention also relates to aqueous liquid composition for lyophilization comprising daptomycin or pharmaceutically acceptable salts or solvates thereof, methyl paraben, and sodium chloride, wherein the pH of the liquid composition is from about 4 to about 9.

The present invention also relates to aqueous liquid composition for lyophilization comprising daptomycin or pharmaceutically acceptable salts or solvates thereof, methyl paraben, sodium chloride, and sodium hydroxide, wherein the pH of the liquid composition is from about 4 to about 9.

The present invention also relates to aqueous liquid composition for lyophilization comprising daptomycin or pharmaceutically acceptable salts or solvates thereof, methyl paraben, sodium chloride and sodium hydroxide, wherein the pH of the liquid composition is about 4.7.

The present invention also relates to a method of manufacturing daptomycin composition comprising the steps of a) forming an aqueous daptomycin solution comprising daptomycin or pharmaceutically acceptable salts or solvates thereof at a pH of about 4 to about 9 and b) lyophilizing the aqueous daptomycin solution.

The present invention also relates to a method of manufacturing daptomycin composition comprising the steps of a) forming an aqueous daptomycin solution comprising daptomycin or pharmaceutically acceptable salts or solvates thereof at a pH of about 8.5 and b) lyophilizing the aqueous daptomycin solution.

The present invention further relates to a method of manufacturing daptomycin composition comprising the steps of a) forming an aqueous daptomycin solution comprising daptomycin or pharmaceutically acceptable salts or solvates thereof and optionally a preservative, pH adjusting agent and/or chaotropic agent at a pH of about 4 to about 9 and b) lyophilizing the aqueous daptomycin solution.

The present invention further relates to a method of manufacturing daptomycin composition comprising the steps of a) forming an aqueous daptomycin solution comprising daptomycin or pharmaceutically acceptable salts or solvates thereof and methyl paraben at a pH of about 4 to about 9 and b) lyophilizing the aqueous daptomycin solution.

The present invention further relates to a method of manufacturing daptomycin composition comprising the steps of a) forming an aqueous daptomycin solution comprising daptomycin or pharmaceutically acceptable salts or solvates thereof, methyl paraben and sodium chloride at a pH of about 4 to about 9 and b) lyophilizing the aqueous daptomycin solution.

The present invention further relates to a method of manufacturing daptomycin composition comprising the steps of a) forming an aqueous daptomycin solution comprising daptomycin or pharmaceutically acceptable salts or solvates thereof, methyl paraben, sodium hydroxide and sodium chloride at a pH of about 4 to about 9 and b) lyophilizing the aqueous daptomycin solution.

### DETAILED DESCRIPTION OF THE INVENTION

The first embodiment of the invention provides lyophilized injectable compositions of daptomycin.

The lyophilized compositions of the presently disclosed invention display improved reconstitution time in a pharmaceutically acceptable diluent(s). As used herein, the term ‘improved reconstitution time’ refers to dissolution of the lyophilized composition in pharmaceutically acceptable reconstitution fluid/diluent in less than 10 minutes. In one embodiment the lyophilized daptomycin compositions of present invention allow for simple and improved reconstitution with a reconstitution time of less than 10 minutes and improved homogeneity making it easily and precisely delivered to a patient. In a further embodiment, the lyophilized daptomycin compositions are reconstituted in a pharmaceutically acceptable diluent in less than about 5 minutes. In another embodiment, the lyophilized daptomycin compositions are reconstituted in a pharmaceutically acceptable diluent in less than about 4 minutes. In a further embodiment, the lyophilized daptomycin compositions are reconstituted in a pharmaceutically acceptable diluent in less than about 3 minutes. In another embodiment, the lyophilized daptomycin compositions are reconstituted in a pharmaceutically acceptable diluent in less than about 2 minutes. In yet another embodiment, the lyophilized daptomycin compositions are reconstituted in a pharmaceutically acceptable diluent in less than about 1 minute. The lyophilized daptomycin compositions of the presently disclosed subject matter have improved reconstitution time compared to CUBICIN® product.

The lyophilized compositions of the present invention contain from about 200 mg to about 2000 mg of daptomycin. In some embodiments the lyophilized compositions include from about 250 mg to about 500 mg of daptomycin. In one particular embodiment, the lyophilized daptomycin compositions include 350 mg of daptomycin. In yet another particular embodiment, the lyophilized daptomycin compositions include 500 mg of daptomycin.

In one of the embodiments, the present invention provides aqueous liquid composition for lyophilization comprising daptomycin or pharmaceutically acceptable salts or solvates thereof and a solvent, wherein the pH of the liquid composition is from about 4 to about 9.

The liquid composition for lyophilization can be lyophilized with any solvent known in the art. Acceptable solvents include, but are not limited to sterile water for injection, aqueous butanol, and aqueous ethanol (96% ethanol).

In another embodiment the invention provides aqueous liquid composition for lyophilization comprising daptomycin or pharmaceutically acceptable salts or solvates thereof and optionally a pH adjusting agent, wherein the pH of the liquid composition is about 8.5.

In another embodiment the invention provides aqueous liquid composition for lyophilization comprising daptomycin or pharmaceutically acceptable salts or solvates thereof and a pH adjusting agent, wherein the pH of the liquid composition is from about 4 to about 9.

In the embodiments of the present invention the pH adjusting agent may be selected from sodium hydroxide, hydrochloric acid, phosphoric acid and/or acetic acid. In a particular embodiment, the pH adjusting agent is sodium hydroxide.

In yet another embodiment the present invention provides lyophilized composition comprising daptomycin or pharmaceutically acceptable salts or solvates thereof and a preservative.

In the embodiments of the invention the preservative may be selected from the group comprising benzyl alcohol, chlorobutanol, m-cresol, methylparaben, phenol, phenoxyethanol, propylparaben, thimerosal, phenylmercuric acetate, phenylmercuric nitrate, benzalkonium chloride, chlorocresol, phenylmercuric salts, and methylhydroxybenzoate or mixtures thereof. In a particular embodiment, the preservative is methyl paraben.

It has been surprisingly observed, that methyl paraben, well known for its preservative action, has demonstrated dissolution-enhancer properties with daptomycin, thereby contributing to its improved reconstitution time.

In the embodiments of the invention the amount of preservative may be conveniently up to 5% based on the weight of daptomycin, more preferably from about 0.01% to about 2% based on the weight of daptomycin and most preferably in an amount of about 1.5% based on the weight of daptomycin.

In one embodiment the present invention provides lyophilized composition comprising daptomycin or pharmaceutically acceptable salts or solvates thereof, a preservative and a pH adjusting agent.

In another embodiment the present invention provides lyophilized composition comprising daptomycin or pharmaceutically acceptable salts or solvates thereof, a preservative and a pH adjusting agent, wherein the pH of the lyophilized composition is from about 4 to about 9.

In a further embodiment the present invention provides lyophilized composition comprising daptomycin or pharmaceutically acceptable salts or solvates thereof, preservative and a pH adjusting agent, wherein the pH of the lyophilized composition is about 4.7.

In another embodiment the present invention provides aqueous liquid composition for lyophilization comprising daptomycin or pharmaceutically acceptable salts or solvates thereof and a preservative.

In another embodiment the present invention provides aqueous liquid composition for lyophilization comprising daptomycin or pharmaceutically acceptable salts or solvates thereof, and a preservative, wherein the pH of the liquid composition is from about 4 to about 9.

In a further embodiment the present invention provides aqueous liquid composition for lyophilization comprising daptomycin or pharmaceutically acceptable salts or solvates thereof, preservative and a pH adjusting agent, wherein the pH of the liquid composition is from about 4 to about 9.

In one embodiment the present invention provides lyophilized composition comprising daptomycin or pharmaceutically acceptable salts or solvates thereof and methyl paraben.

In another embodiment the present invention provides lyophilized composition comprising daptomycin or pharmaceutically acceptable salts or solvates thereof, methyl paraben and sodium hydroxide.

In the embodiments of the invention the amount of methyl paraben may be conveniently up to 5% based on the weight of daptomycin, more preferably from about 0.01% to about 2% based on the weight of daptomycin and most preferably in an amount of about 1.5% based on the weight of daptomycin.

In one embodiment the present invention provides aqueous liquid composition for lyophilization comprising daptomycin or pharmaceutically acceptable salts or solvates thereof and methyl paraben.

In a further embodiment the present invention provides aqueous liquid composition for lyophilization comprising daptomycin or pharmaceutically acceptable salts or solvates thereof, and methyl paraben, wherein the pH of the liquid composition is from about 4 to about 9.

In another embodiment the present invention provides aqueous liquid composition for lyophilization comprising daptomycin or pharmaceutically acceptable salts or solvates thereof, and methyl paraben, wherein the pH of the liquid composition is about 4.7.

In another embodiment the present invention provides aqueous liquid composition for lyophilization comprising daptomycin or pharmaceutically acceptable salts or solvates thereof, methyl paraben and sodium hydroxide, wherein the pH of the liquid composition is from about 4 to about 9.

In further embodiment the present invention provides aqueous liquid composition for lyophilization comprising daptomycin or pharmaceutically acceptable salts or solvates thereof, methyl paraben and sodium hydroxide, wherein the pH of the liquid composition is about 4.7.

In yet another embodiment the present invention provides lyophilized composition comprising daptomycin or pharmaceutically acceptable salts or solvates thereof, preservative and a chaotropic agent.

As used herein “chaotropic agent” refers to a compound that is capable of changing the state of hydration of daptomycin and which enhances the solubility of daptomycin in aqueous medium.

In the embodiments of the invention chaotropic agents are selected from the group comprising sodium chloride, potassium chloride, magnesium chloride, zinc chloride and calcium chloride.

In the embodiments of the invention the amount of chaotropic agent may be conveniently up to 50% based on the weight of daptomycin, more preferably from about 1% to about 40% based on the weight of daptomycin and most preferably in an amount of about 3% to about 25% based on the weight of daptomycin.

In one embodiment the present invention provides lyophilized composition comprising daptomycin or pharmaceutically acceptable salts or solvates thereof, preservative, chaotropic agent and a pH adjusting agent.

In another embodiment the present invention provides lyophilized composition comprising daptomycin or pharmaceutically acceptable salts or solvates thereof, preservative, chaotropic agent and a pH adjusting agent, wherein the pH of the lyophilized composition is from about 4 to about 9.

In a further embodiment the present invention provides lyophilized composition comprising daptomycin or pharmaceutically acceptable salts or solvates thereof, preservative, chaotropic agent and a pH adjusting agent, wherein the pH of the lyophilized composition is from about 4.7.

In one embodiment the present invention provides aqueous liquid composition for lyophilization comprising daptomycin or pharmaceutically acceptable salts or solvates thereof, preservative and a chaotropic agent.

In a further embodiment the present invention provides aqueous liquid composition for lyophilization comprising daptomycin or pharmaceutically acceptable salts or solvates thereof, preservative in an amount of up to 5% based on the weight of daptomycin and a chaotropic agent in amount of up to 50% based on the weight of daptomycin.

In another embodiment the present invention provides aqueous liquid composition for lyophilization comprising daptomycin or pharmaceutically acceptable salts or solvates thereof, preservative and a chaotropic agent, wherein the pH of the liquid composition is from about 4 to about 9.

In another embodiment the present invention provides aqueous liquid composition for lyophilization comprising daptomycin or pharmaceutically acceptable salts or solvates thereof, preservative and a chaotropic agent, wherein the pH of the liquid composition is about 4.7.

In one embodiment the present invention provides aqueous liquid composition for lyophilization comprising daptomycin or pharmaceutically acceptable salts or solvates thereof, preservative, chaotropic agent and a pH adjusting agent, wherein the pH of the liquid composition is from about 4 to about 9.

The another embodiment the present invention provides aqueous liquid composition for lyophilization comprising daptomycin or pharmaceutically acceptable salts or solvates thereof, preservative, chaotropic agent and a pH adjusting agent, wherein the pH of the liquid composition is about 4.7.

In one embodiment the present invention provides lyophilized composition comprising daptomycin or pharmaceutically acceptable salts or solvates thereof, methyl paraben and sodium chloride.

In another embodiment the present invention provides lyophilized composition comprising daptomycin or pharmaceutically acceptable salts or solvates thereof, methyl paraben in an amount of up to 5% based on the weight of daptomycin and sodium chloride in amount of up to 50% based on the weight of daptomycin.

In another embodiment the present invention provides lyophilized composition comprising daptomycin or pharmaceutically acceptable salts or solvates thereof, preservative in an amount from about 0.01% to about 2% based on the weight of daptomycin and chaotropic agent in an amount from about 3% to about 25% based on the weight of daptomycin.

In a further embodiment the present invention provides lyophilized composition comprising daptomycin or pharmaceutically acceptable salts or solvates thereof, methyl paraben, sodium chloride and sodium hydroxide.

In another embodiment the present invention provides lyophilized composition comprising daptomycin or pharmaceutically acceptable salts or solvates thereof, methyl paraben in an amount of up to 5% based on the weight of daptomycin, sodium chloride in amount of up to 50% based on the weight of daptomycin and sodium hydroxide.

In another embodiment the present invention provides lyophilized composition comprising daptomycin or pharmaceutically acceptable salts or solvates thereof, methyl paraben in an amount from about 0.01% to about 2% based on the weight of daptomycin, sodium chloride in an amount from about 3% to about 25% based on the weight of daptomycin and sodium hydroxide.

In another embodiment the present invention provides aqueous liquid composition for lyophilization comprising daptomycin or pharmaceutically acceptable salts or solvates thereof, methyl paraben and sodium chloride.

In a further embodiment the present invention provides aqueous liquid composition for lyophilization comprising daptomycin or pharmaceutically acceptable salts or solvates thereof, methyl paraben in an amount of up to 5% based on the weight of daptomycin and a sodium chloride in amount of up to 50% based on the weight of daptomycin.

In a further embodiment the present invention provides aqueous liquid composition for lyophilization comprising daptomycin or pharmaceutically acceptable salts or solvates thereof, methyl paraben in an amount from about 0.01% to about 2% based on the weight of daptomycin and sodium chloride in an amount from about 3% to about 25% based on the weight of daptomycin.

In one embodiment the present invention provides aqueous liquid composition for lyophilization comprising daptomycin or pharmaceutically acceptable salts or solvates thereof, methyl paraben, and sodium chloride, wherein the pH of the liquid composition is from about 4 to about 9.

In a further embodiment the present invention provides aqueous liquid composition for lyophilization comprising daptomycin or pharmaceutically acceptable salts or solvates thereof, methyl paraben, sodium chloride, and sodium hydroxide, wherein the pH of the liquid composition is from about 4 to about 9.

In another embodiment the present invention provides aqueous liquid composition for lyophilization comprising daptomycin or pharmaceutically acceptable salts or solvates thereof, methyl paraben, sodium chloride and sodium hydroxide, wherein the pH of the liquid composition is about 4.7

Further optional excipients can be included in aqueous liquid compositions of the present invention which include but are not limited to sugars, antioxidants, organic acids, buffering agents and/or complexing agents.

Suitable sugars may be selected from the group comprising sucrose, mannitol, trehalose or combinations thereof.

Suitable antioxidants may be selected from the group comprising ascorbic acid, monothioglycerol, L-cysteine, thioglycolic acid, sodium metabisulfite, sodium EDTA, sodium formaldehyde sulfoxylate and sodium bisulfite.

Suitable organic acids may be selected from the group comprising monocarboxylic organic acids, dicarboxylic organic acids, hydroxyl substituted dicarboxylic organic acids, tricarboxylic organic acids, and hydroxyl substituted tricarboxylic organic acids, tetra carboxylic organic acids, or combinations thereof. In one embodiment the hydroxyl substituted tricarboxylic organic acid is citric acid.

Suitable buffering agents may be selected from the group comprising TRIS (tris (hydroxymethyl) aminomethane, salts of maleic acid, sodium or potassium salt of phosphoric acid, a sodium or potassium salt of boric acid, a sodium or potassium salt of citric acid, a sodium or potassium salt of carbonic acid and sodium phosphate (e.g., Sodium phosphate dibasic).

Suitable complexing agents may be selected from the group comprising cyclodextrin derivatives including α-cyclodextrin, β-cyclodextrin, γ-cyclodextrin, modified α-cyclodexin, modified β-cyclodextin and modified γ-cyclodextrin and a combination thereof, preferably modified β-cyclodextin, i.e. hydroxypropyl β-cyclodextrin, sulfobutyl ether β-cyclodextrin, and randomly methylated β-cyclodextrin.

The embodiments of the invention also provide method for manufacturing said daptomycin compositions.

In one embodiment the invention provides a method of manufacturing daptomycin composition comprising the steps of a) forming an aqueous daptomycin solution comprising daptomycin or pharmaceutically acceptable salts or solvates thereof at a pH of about 4 to about 9 and b) lyophilizing the aqueous daptomycin solution.

In a further embodiment the invention provides a method of manufacturing daptomycin composition comprising the steps of a) forming an aqueous daptomycin solution comprising daptomycin or pharmaceutically acceptable salts or solvates thereof and optionally a pH adjusting agent, wherein the pH of the solution is about 8.5 and b) lyophilizing the aqueous daptomycin solution.

In one embodiment, the invention provides a method of manufacturing daptomycin composition comprising the steps of a) forming an aqueous liquid composition for lyophilization comprising daptomycin or pharmaceutically acceptable salts or solvates thereof, a preservative, and optionally a pH adjusting agent, a chaotropic agent and adjusting the pH to about 4 to about 9; and b) lyophilizing the solution of step a).

In yet another embodiment, the invention provides a method of manufacturing daptomycin composition comprising the steps of a) forming an aqueous liquid composition for lyophilization comprising daptomycin or pharmaceutically acceptable salts or solvates thereof, methyl paraben, and sodium hydroxide, and b) lyophilizing the solution of step a).

In another embodiment the invention provides a method of manufacturing daptomycin composition comprising the steps a) forming an aqueous daptomycin solution comprising daptomycin or pharmaceutically acceptable salts or solvates thereof and sodium hydroxide, wherein the pH of aqueous daptomycin solution is about 8.5 and b) lyophilizing the aqueous daptomycin solution.

In one embodiment the invention provides a method of manufacturing daptomycin composition comprising the steps a) forming an aqueous daptomycin solution comprising daptomycin or pharmaceutically acceptable salts or solvates thereof and optionally a pH adjusting agent, a preservative and/or chaotropic agent wherein the pH of aqueous daptomycin solution is about 4 to about 9 and b) lyophilizing the aqueous daptomycin solution.

In one embodiment the invention provides a method of manufacturing daptomycin composition comprising the steps a) forming an aqueous daptomycin solution comprising daptomycin or pharmaceutically acceptable salts or solvates thereof and optionally sodium hydroxide, methyl paraben and/or sodium chloride wherein the pH of aqueous daptomycin solution is about 4 to about 9 and b) lyophilizing the aqueous daptomycin solution. In a particular embodiment, the pH of the solution is adjusted to 4.7.

In the embodiments of present invention lyophilization is done according to procedures well known in the art. Lyophilization is a stabilizing process in which a substance is first frozen (freezing) and then the quantity of the solvent is reduced, first by sublimation (referred to as the primary drying process) and then desorption (known as the secondary drying process) to values that will no longer support chemical reactions. In the embodiments of the present invention the lyophilization process comprises one or more annealing steps during freezing. The annealing step is done at a temperature range between −5° C. and −30° C. during the freezing.

As used herein, ‘annealing’ is defined as process of transient increase in product temperature from initial set point to higher or lower set point, and then bringing the product temperature back to original set point.

The lyophilized compositions of daptomycin of the present invention can be contained within a sealed container. More preferably, the container is provided with an opening and means for aseptically sealing the opening, such that the sealed container is fluidly sealed or the sealed opening is substantially impermeable to atmospheric gases, moisture, pathogenic microorganisms or the like. The container can be constructed with any suitable material such as, glass, polypropylene, polyethylene terephthalate, and the like. In a preferred embodiment the container is constructed of glass. Suitable glass vials include molded and tubing glass vials such as, Type I molded glass vials, and the like.

A suitable means for sealing the container can include, for example, a stopper, a cap, a lid, a closure, a covering which fluidly seals the container, or the like. The means for sealing the container are not limited to separate closures or closure devices. In a preferred embodiment, the means for aseptically sealing the container includes a stopper such as, a stopper that is configured to fluidly seal the opening. Suitable stoppers include conventional medical grade stoppers which do not degrade or release significant amounts of impurities upon exposure to the constituted daptomycin formulation. Preferably, the stopper is constructed of an elastomer, which is more preferably an elastomer that is pierceable by a hypodermic needle or a blunt cannula. Exemplary stoppers include bromobutyl stoppers.

Optionally, an outer seal is provided which covers and entirely surrounds the stopper. The outer seal can be constructed of any suitable material. When an outer seal is used, it is preferably fitted with a lid that can be easily manually removed to provide access to the stopper. Suitable outer seals can include, for example, Flip-off Aluminum/Polypropylene Seals. Such seals include an outer rim made of a suitable material, such as aluminum, that entirely surrounds the lateral edge of the stopper and further include a lid (typically polypropylene or other suitable material) that entirely covers the upper surface of the stopper. The polypropylene lid can be “flipped” off e.g., by exerting upward pressure with a finger or thumb, to provide access to the stopper, e.g., so that it can be punctured with a hypodermic needle to deliver an aqueous vehicle for reconstitution.

The one or more containers preferably include one or more sterile vials, preferably glass vials, as described herein. The sealing step preferably includes sealing the opening using the means for aseptically sealing the opening described herein. The sealing means preferably includes a stopper as described herein.

The lyophilized daptomycin compositions of the present invention can be reconstituted with one or more pharmaceutically acceptable diluents to provide a solution suitable for administration. The pharmaceutically acceptable diluents include, but are not limited to, sterile water for injection, bacteriostatic water for injection, 0.45% sodium chloride solution for injection and 0.9% sodium chloride solution for injection, Ringer's solution and lactated Ringer's solution. In one embodiment, the lyophilized daptomycin compositions of the present invention are reconstituted with 0.9% sterile sodium chloride solution for injection.

The pharmaceutically acceptable diluent optionally comprises one or more pharmaceutically acceptable excipients as described above. Preferably, the diluent comprises an organic acid selected from the group consisting of citric acid, tartaric acid, succinic acid, edetic acid, lactic acid, acetic acid, malonic acid, butyric acid and the like.

According to one embodiment, the present invention includes pharmaceutical compositions presented as co-pack, comprising two vials, wherein one vial contains the lyophilized injectable daptomycin composition of the present invention and the other vial contains a pharmaceutically acceptable reconstitution diluent.

The lyophilized daptomycin compositions of the present invention can be reconstituted by adding the pharmaceutically acceptable diluent(s) to the lyophilized daptomycin formulation to provide the desired concentration for direct administration or further dilution for administration by infusion. In one embodiment, the volume of the pharmaceutically acceptable diluent(s) added to the lyophilized daptomycin formulation is about 10 mL.

The lyophilized daptomycin compositions of the present invention can be reconstituted by any suitable methods known to one of ordinary skill in the art. For example, 10 mL of 0.9% sterile sodium chloride for injection is added slowly to a vial including 500 mg of the lyophilized daptomycin formulation. The resultant mixture is rotated to ensure all of the formulation is wetted and then allowed to stand undisturbed for about 2 minutes. The vial is then gently rotated or swirled intermittently as needed, to obtain a completely reconstituted solution. Additionally and alternatively, the reconstitution method includes quickly adding a diluent to a vessel including a lyophilized daptomycin formulation of the presently disclosed subject matter, followed by swirling of the vessel if required.

The reconstituted formulation of the present invention will have the daptomycin concentration of about 20 mg/mL to about 200 mg/mL, preferably 50 mg/mL. The reconstituted daptomycin formulation can be further diluted in a pharmaceutically acceptable diluent for administration to a subject. Pharmaceutically acceptable diluents include, but are not limited to, sterile water for injection, bacteriostatic water for injection, 0.9% sodium chloride solution for injection, Ringer's solution and lactated Ringer's solution. In one embodiment, when the reconstituted daptomycin formulation is further diluted for administration to a subject, the final daptomycin concentration is from about 1 mg/mL to about 20 mg/mL.

Embodiments of the present invention relates to the usage of lyophilized compositions for treatment of complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only) and Staphylococcus aureus bloodstream infections (bacteremia), including those with right-sided infective endocarditis, caused by methicillin-susceptible and methicillin-resistant isolates.

The following examples further describe certain specific embodiments of the invention and demonstrate the practice and advantages thereof. It is to be understood that the examples are given by way of illustration only and are not intended to limit the scope of the invention in any manner.

### EXAMPLES

### Example 1

### Composition:

| Ingredient | Quantity per vial |
|---|---|
| Daptomycin | 500 mg |
| Sodium hydroxide | Q.S to pH 8.5 |
| Sterile Water for Injection^{¥} | Q.S to 5 mL |

^{¥}Removed during lyophilization

### Brief Manufacturing Process

1.70% of total required quantity of sterile water for injection was taken and purged with nitrogen and then cooled to 2-8° C.
2. Daptomycin was added to water of step 1 and dissolved by stirring for 20 minutes.
3. The pH was adjusted to 8.5 using 2N NaOH.
4. Volume was adjusted to 100% with the rest 30% of sterile water for injection and stirred for 10 minutes.
5. 5 mL of the above solution was filled in 15 mL clear glass vials, stoppered partially and loaded for lyophilization as per the lyophilization cycle below.

| | Temperature | Vacuum | Ramp | Hold |
|---|---|---|---|---|
| Freezing | −5° C. | — | 1° C./min | 120 min |
| | −30° C. | — | 1° C./min | 140 min |
| 1° C. Drying | −10° C. | 50 mTorr | 1° C./min | 1200 min |
| 2° C. Drying | 5° C. | 50 mTorr | 1° C./min | 240 min |

6. After completion of lyophilization cycle, vials were stoppered under partial vacuum of 2,50000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared as above and the resultant mixture was allowed to stand. The reconstitution time of the vial was found to be about 3-5 minutes.

### Example 2

### Composition:

| Ingredient | Quantity per vial |
|---|---|
| Daptomycin | 500 mg |
| Hydroxy propyl-β-cyclodextrin | 0.4% |
| Sodium hydroxide | Q.S to pH 4.7 |
| Sterile Water for Injection^{¥} | Q.S to 5 mL |

^{¥}Removed during lyophilization

### Brief Manufacturing Process

1. 80% of total required quantity of sterile water for injection was taken and purged with nitrogen and then cooled to 2-8° C.
2. Hydroxy propyl-β-cyclodextrin was added to the water of step 1 and dissolved for 10 minutes.
3. Daptomycin was then added and dissolved by stirring for 30 minutes.
4. The pH was adjusted to 4.7 using 0.5N NaOH.
5. Volume was adjusted to 100% with the rest 20% of sterile water for injection and stirred for 10 minutes.
6. 5 mL of the above solution was filled in 15 mL clear glass vials, stoppered partially and loaded for lyophilization as per the lyophilization cycle below.

| | Temperature | Vacuum | Ramp | Hold |
|---|---|---|---|---|
| Freezing | −5° C. | — | 1° C./min | 120 min |
| | −30° C. | — | 1° C./min | 140 min |
| 1° C. Drying | −10° C. | 50 mTorr | 1° C./min | 1200 min |
| 2° C. Drying | 5° C. | 50 mTorr | 1° C./min | 240 min |

7. After completion of lyophilization cycle, vials were stoppered under partial vacuum of 2,50000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared in example 2 and the resultant mixture was allowed to stand. The reconstitution time of vial in was found to be about 3 minutes.

### Examples 3 & 4

### Composition:

Quantity per vial

| Ingredient | Example-3 | Example-4 |
|---|---|---|
| Daptomycin | 500 mg | 500 mg |
| 96% Ethanol^{¥} | 2% (0.1 mL) | 5% (0.25 mL) |
| Water for Injection^{¥} | Q.S to 5 mL | Q.S to 5 mL |
| Sodium hydroxide | Q.S to pH 4.7 | Q.S to pH 4.7 |

^{¥}Removed during lyophilization

### Brief Manufacturing Process

1. Ethanol solutions 2% and 5% (respectively for examples 3 and 4) were prepared by dissolving 0.1 mL and 0.25 mL of 96% ethanol in Q.S for 5 mL of water for Injection.
2. 40% of required solvent solution of step 1 of was transferred into a beaker, purged with nitrogen and cooled to 2° C.-8° C.
3. Daptomycin was added to the above solution and stirred for 20 minutes and the pH of the solution was adjusted to 4.7 using 0.5N NaOH.
4. Volume was adjusted to 100% with the remaining ethanolic solutions (2% and 5% for example-3 and example-4 respectively) and stirred for 10 minutes.
5. 5 mL of the above solution was filled in 15 mL clear glass vials, stoppered partially and loaded for lyophilization as per the lyophilization cycle below.

| | Temperature | Vacuum | Ramp | Hold |
|---|---|---|---|---|
| Freezing | −5° C. | — | 1° C./min | 120 min |
| | −30° C. | — | 1° C./min | 140 min |
| 1° C. Drying | −10° C. | 50 mTorr | 1° C./min | 1200 min |
| 2° C. Drying | 5° C. | 50 mTorr | 1° C./min | 240 min |

6. After completion of lyophilization cycle, vials were stoppered under partial vacuum of 2,50000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared in example-3 & 4 and the resultant mixture was allowed to stand. The reconstitution time of vial in both examples 3 & 4 was found to be about 6 minutes.

### Example-5

### Composition:

| Ingredient | Quantity per vial |
|---|---|
| Daptomycin | 500 mg |
| Methyl paraben | 7.5 mg |
| Sodium hydroxide | Q.S to pH 4.7 |
| Sterile Water for Injection^{¥} | Q.S to 5 mL |

^{¥}Removed during lyophilization

### Brief Manufacturing Process

The above composition was lyophilized using two procedures.

### Procedure-1:—

1. 70% of total required quantity of sterile water for injection was taken and purged with nitrogen.
2. Methyl paraben was added and dissolved by heating at 40° C. for 10 min and the solution was stirred for 30 min.
3. The above solution was cooled to 2-8° C. and daptomycin was added by stirring for 20 min and the pH of the solution was adjusted to 4.7 using 0.5N NaOH.
4. Volume was adjusted to 100% with the rest 30% of sterile water for injection and stirred for 10 minutes.
5. 5 mL of the solution was filled in each 15 mL clear glass vial stoppered partially and loaded for lyophilization cycle as per below cycle

| | Temperature | vacuum | Ramp | Hold |
|---|---|---|---|---|
| Freezing | −30° C. | — | 1° C./min | 140 min |
| 1° C. Drying | −10° C. | 50 mTorr | 1° C./min | 1800 min |
| 2° C. Drying | 5° C. | 50 mTorr | 1° C./min | 600 min |

6. After completion of lyophilization cycle vials were stoppered under partial vacuum of 2, 50,000-4,50000 mTorr and sealed.

### Procedure-2:—

1. 70% of total required quantity of sterile water for injection was taken and purged with nitrogen.
2. Methyl paraben was added to the water of step 1 and dissolved by heating at 40° C. for 10 minutes and the solution was stirred for 30 minutes.
3. The above solution was cooled to 2-8° C. and daptomycin was added and dissolved by stirring for 20 minutes and the pH of the solution was made up to 4.7 using 0.5N NaOH.
4. Volume was adjusted to 100% with rest 30% of sterile water for injection and stirred for 10 minutes.
5. 5 mL of the solution was filled in each 15 mL clear glass vial stoppered partially and loaded for lyophilization cycle as per below cycle.

| | Temperature | Vacuum | Ramp | Hold |
|---|---|---|---|---|
| Freezing | −5° C. | — | 1° C./min | 120 min |
| | −30° C. | — | 1° C./min | 140 min |
| 1° C. Drying | −10° C. | 50 mTorr | 1° C./min | 1200 min |
| 2° C. Drying | 5° C. | 50 mTorr | 1° C./min | 240 min |

6. After completion of lyophilization cycle vials were stoppered under partial vacuum of 2,50,000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared as above and the resultant mixture was allowed to stand. The reconstitution time of the above composition using procedure-1 and procedure-2 was found to be about 2.5 and 2 minutes respectively.

### Example-6 & 7

### Composition:

Quantity Per Vial

| Ingredient | Example-6 | Example-7 |
|---|---|---|
| Daptomycin | 500 mg | 500 mg |
| Methyl paraben | 7.5 mg | 7.5 mg |
| Sodium Chloride | 90 | 20 |
| Sodium hydroxide | Q.S to pH 4.7 | Q.S to pH 4.7 |
| Sterile Water for Injection^{¥} | Q.S to 5 mL | Q.S to 5 mL |

^{¥}Removed during lyophilization

### Brief Manufacturing Process

1. 80% of total required quantity of sterile water for injection containing required quantity of sodium chloride was taken and purged with nitrogen for one hour.
2. Methyl paraben was added to the solutions of step 1 and dissolved by heating at 40° C. for 10 minutes and the solution was stirred for 10 minutes.
3. The above solution was cooled to 2-8° C. and daptomycin was added by stirring for 20 minutes and the pH of the solution was adjusted to 4.7 using 0.5N NaOH.
4. Volume was adjusted to 100% with the rest 20% of sterile water for injection and stirred for 10 minutes.
5. 5 mL of the solution was filled in each 15 mL clear glass vial stoppered partially and loaded for lyophilization cycle as per below cycle.

Lyophilization Cycle for Example-6

| | Temperature | vacuum | Ramp | Hold |
|---|---|---|---|---|
| Freezing | −40° C. | — | 1° C./min | — |
| | −10° C. | — | 1° C./min | 240 min |
| | −40° C. | — | 1° C./min | 180 min |
| 1° C. Drying | −10° C. | 50 mTorr | 1° C./min | 1800 min |
| 2° C. Drying | 5° C. | 50 mTorr | 1° C./min | 300 min |

Lyophilization Cycle for Example-7

| | Temperature | vacuum | Ramp | Hold |
|---|---|---|---|---|
| Freezing | −10° C. | — | 1° C./min | 120 min |
| | −30° C. | — | 1° C./min | 140 min |
| 1° C. Drying | −10° C. | 50 mTorr | 1° C./min | 1800 min |
| 2° C. Drying | 5° C. | 50 mTorr | 1° C./min | 280 min |

6. After completion of lyophilization cycle vials were stoppered under partial vacuum of 2, 50000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared as above and the resultant mixture was allowed to stand. The reconstitution time of vial in examples 6 was found to be less than one minute and in Example-7 was found to be 3 minutes.

### Example 8

### Composition:

| Ingredient | Quantity per vial |
|---|---|
| Daptomycin | 500 mg |
| Methyl paraben | 7.14 mg |
| Sodium hydroxide | Q.S to pH 4.7 |
| Sterile Water for Injection^{¥} | Q.S to 7.14 mL |

^{¥}Removed during lyophilization

### Brief Manufacturing Process

1. 70% of total required quantity of sterile water for injection was taken and purged with nitrogen.
2. Methyl paraben was added to the water of step 1 and dissolved by heating at 60° C. for 10 minutes and the solution was stirred for 30 minutes.
3. The above solution was cooled to 2-8° C. and daptomycin was added by stirring for 20 minutes and the pH of the solution was adjusted to 4.7 using 0.5N NaOH.
4. Volume was adjusted to 100% with the rest 30% of sterile water for injection and stirred for 10 minutes.
5. 7.14 mL of the solution was filled in each 15 mL clear glass vial stoppered partially and loaded for lyophilization cycle as per below cycle

| | Temperature | vacuum | Ramp | Hold |
|---|---|---|---|---|
| Freezing | −30° C. | — | 1° C./min | — |
| | −0° C. | — | 1° C./min | 240 min |
| | −40° C. | — | 1° C./min | 180 min |
| 1° C. Drying | −10° C. | 50 mTorr | 1° C./min | 2100 min |
| 2° C. Drying | 5° C. | 50 mTorr | 1° C./min | 300 min |

6. After completion of lyophilization cycle, vials were stoppered under partial vacuum of 2,50,000-4,50000 mTorr and sealed.
   Reconstitution of Lyophilized Formulation with 0.9% Sodium Chloride

Reconstitution time was determined by injecting 10 mL of 0.9% sodium chloride slowly into center of the vial of 500 mg of daptomycin formulation prepared as above and the resultant mixture was allowed to stand. The reconstitution time of the vial was found to be about 2 minutes.

### Example 9

### Composition:

| Ingredient | Quantity per vial |
|---|---|
| Daptomycin | 350 mg |
| Methyl paraben | 5.0 mg |
| Sodium hydroxide | Q.S to pH 4.7 |
| Sterile Water for Injection^{¥} | Q.S to 5.0 mL |

^{¥}Removed during lyophilization

### Brief Manufacturing Process

### Procedure-1:

1. 70% of total required quantity of sterile water for injection was taken and purged with nitrogen.
2. Methyl paraben was added and dissolved by heating at 60° C. for 10 min and the solution was stirred for 30 min.
3. The above solution was cooled to 2-8° C. and daptomycin was added by stirring for 20 min and the pH of the solution was adjusted to 4.7 using 0.5N NaOH.
4. Volume was adjusted to 100% with the rest 30% of sterile water for injection and stirred for 10 minutes.
5. 5.0 mL of the solution was filled in each 15 mL clear glass vial stoppered partially and loaded for lyophilization cycle as per below cycle

| | Temperature | vacuum | Ramp | Hold |
|---|---|---|---|---|
| Freezing | −30° C. | — | 1° C./min | — |
| | −0° C. | — | 1° C./min | 240 min |
| | −30° C. | — | 1° C./min | 180 min |
| 1° C. Drying | −10° C. | 50 mTorr | 1° C./min | 1800 min |
| 2° C. Drying | 5° C. | 50 mTorr | 1° C./min | 300 min |

6. After completion of lyophilization cycle vials were stoppered under partial vacuum of 2, 50,000-4,50000 mTorr and sealed.
   Reconstitution of Lyophilized Formulation with 0.9% Sodium Chloride

Reconstitution time was determined by injecting 7 mL of 0.9% sodium chloride slowly into center of the vial containing 350 mg of daptomycin formulation prepared as above and the resultant mixture was allowed to stand. The reconstitution time of the vial was found to be about 2.5 minutes.

1.-14. (canceled)

15. A lyophilized injectable composition comprising daptomycin or pharmaceutically acceptable salts or solvates thereof and a preservative, wherein said composition has an improved reconstitution time.

16. The lyophilized injectable composition according to claim 15, wherein the preservative is selected from the group comprising of benzyl alcohol, chlorobutanol, m-cresol, methylparaben, phenol, phenoxyethanol, propylparaben, thimerosal, phenylmercuric acetate, phenylmercuric nitrate, benzalkonium chloride, chlorocresol, phenylmercuric salts, and mixtures thereof.

17. The lyophilized injectable composition according to claim 16, wherein the preservative is methylparaben.

18. The lyophilized injectable composition according to claim 15, wherein the preservative is present in an amount from about 0.01% to about 2% based on the weight of daptomycin.

19. The lyophilized injectable composition according to claim 15, wherein composition comprises from about 200 mg to about 600 mg of daptomycin.

20. The lyophilized injectable composition according to claim 15, further comprising a pH adjusting agent.

21. The lyophilized injectable composition according to claim 20, wherein the pH adjusting agent is selected from the group comprising of sodium hydroxide, hydrochloric acid, phosphoric acid and acetic acid.

22. The lyophilized injectable composition according to claim 21, wherein the pH adjusting agent is sodium hydroxide.

23. The lyophilized injectable composition according to claim 20, wherein the pH of the liquid composition for lyophilization is from about 4 to about 9.

24. The lyophilized injectable composition according to claim 15, further comprising a chaotropic agent, wherein the chaotropic agent is present in an amount from about 1% to about 40% based on the weight of daptomycin.

25. The lyophilized injectable composition according to claim 24, wherein the chaotropic agent is selected from the group comprising of sodium chloride, potassium chloride, magnesium chloride, zinc chloride and calcium chloride.

26. A method of manufacturing a lyophilized injectable composition of daptomycin, wherein the method comprises the steps of:
a) forming an aqueous solution comprising daptomycin or pharmaceutically acceptable salts or solvates thereof and a preservative, and optionally a pH adjusting agent and/or chaotropic agent; and
b) lyophilizing the aqueous daptomycin solution of step a).

27. The method of manufacturing the lyophilized injectable composition of daptomycin according to claim 26, wherein the preservative is selected from the group comprising of benzyl alcohol, chlorobutanol, m-cresol, methylparaben, phenol, phenoxyethanol, propylparaben, thimerosal, phenylmercuric acetate, phenylmercuric nitrate, benzalkonium chloride, chlorocresol, phenylmercuric salts, and mixtures thereof.

28. The method of manufacturing the lyophilized injectable composition of daptomycin according to claim 26, wherein the pH adjusting agent is selected from the group comprising of sodium hydroxide, hydrochloric acid, phosphoric acid and acetic acid.

29. The method of manufacturing the lyophilized injectable composition of daptomycin according to claim 26, wherein the chaotropic agent is selected from the group comprising of sodium chloride, potassium chloride, magnesium chloride, zinc chloride and calcium chloride.

30. The method of manufacturing a lyophilized injectable composition of daptomycin according to claim 26, wherein the preservative is methyl paraben, the pH adjusting agent is optionally sodium hydroxide and/or the chaotropic agent is sodium chloride, and wherein the pH of aqueous solution is ranging from about 4 to about 9.

31. The injectable composition according to claim 26, wherein the composition has reconstitution time less than about 10 minutes.

32. The injectable composition according to claim 31, wherein the reconstitution time less than about 5 minutes.

33. An aqueous liquid injectable composition comprising daptomycin or pharmaceutically acceptable salts or solvates thereof and a preservative, and optionally a pH adjusting agent and/or chaotropic agent, wherein the pH of said composition is from about 4 to about 9.

34. The injectable composition according to claim 15 for the treatment of complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only) and Staphylococcus aureus bloodstream infections (bacteremia), including those with right-sided infective endocarditis, caused by methicillin-susceptible and methicillin-resistant isolates.

35. The injectable composition according to claim 33 for the treatment of complicated skin and skin structure infections (cSSSI) caused by susceptible isolates of the following Gram-positive bacteria: Staphylococcus aureus (including methicillin-resistant isolates), Streptococcus pyogenes, Streptococcus agalactiae, Streptococcus dysgalactiae subsp. equisimilis, and Enterococcus faecalis (vancomycin-susceptible isolates only) and Staphylococcus aureus bloodstream infections (bacteremia), including those with right-sided infective endocarditis, caused by methicillin-susceptible and methicillin-resistant isolates.
